(19) United States
(12) Patent Application Publication
(10) Pub. No.: US 2006/0034948 A1
(43) Pub. Date: Feb. 16, 2006

(54) Method for treating an environment that may be or is contaminated with an undesirable bacterial, virus and or spore

(75) Inventor: Lary D Rogers, Carson City, NV (US)

Correspondence Address: CHRISTOPHER J. KULISH, ESQ, HOLLAND & HART LLP, P. O. BOX 8749, DENVER, CO, 80201-8749, US

(73) Assignee: Sequoia Pacific Research Company, L.L.C., Draper, UT (US)

(21) Appl. No.: 10/529,427

(22) PCT Filed: Oct. 10, 2003
(86) PCT No.: PCT/US03/32267

§ 371 Date: Mar. 25, 2005

Related U.S. Application Data
(60) Provisional application No. 60/417,593, filed on Oct. 10, 2002.

(51) Int. Cl. A61K 33/00

(52) U.S. Cl. 424/722

(57) ABSTRACT

The invention is directed to a method of decontaminating environments that are suspected of or known to be contaminated with undesirable biological agents, i.e., bacteria, viruses and/or spores, using nano-crystals that penetrate the cell wall of the undesirable agent to neutralize the agent. In one embodiment, the method comprises providing a dry compound that, after being mixed with water to form a slurry and a reduction in the temperature of the water in the slurry, forms nano-crystals. The method further comprises mixing the dry compound with water to produce the slurry and applying the slurry to an environment that is suspected of or known to be contaminated with an undesirable biological agent.

### FIELD OF THE INVENTION

The present invention relates to a method for neutralizing an undesirable bacteria, viruses and/or spores that may be present within an environment.

### BACKGROUND OF THE INVENTION

Presently, most of the known methods for neutralizing undesirable bacteria, viruses and/or spores (hereinafter collectively referred to as “undesirable biological agents”) that are present in an environment (i.e., airborne, waterborne or resident on a surface) operate by applying a neutralizing agent to the undesirable biological agent that is toxic to the undesirable biological agent. The use of these toxic, neutralizing agents to rid an environment of undesirable biological agents has several drawbacks. For instance, some of these neutralizing agents, while ostensibly ridding the environment of interest of the undesirable biological agent, are also toxic to humans, animals and/or plant life. In such situations, the treated environment must either remain unoccupied and/or uncultivated for an extended period of time or further remedial action must be taken to neutralize the toxic material. Other neutralizing agents must be in contact with the undesired biological agent for an extended period of time to be effective. In environments that are exposed to the elements (e.g., wind, rain etc.), such neutralizing agents may be removed from the environment before a sufficient amount of time has elapsed to neutralize the undesired biological agent.

### SUMMARY OF THE INVENTION

The present invention is directed to a method of treating an environment that is suspected of being contaminated or known to be contaminated with an undesirable biological agent, such as anthrax. In contrast to the known methods of treating such an environment with a toxic neutralizing agent, the present invention utilizes a mechanical mechanism, namely, a nano-crystal to penetrate (e.g., cut, puncture, tear, rupture, slice, dice and the like) the cell wall of the undesirable biological agent and thereby neutralize the undesirable biological agent. The nano-crystals have sharp edges and/or points that facilitate the penetration of the cell wall of the undesirable bacteria, virus or spore. Further, nano-crystals have an affinity for the water that is present in bacteria, viruses and spores and, as a consequence, naturally adhere to any bacteria, virus or spore with which they come into contact. Further, viruses also have an affinity for crystal structures that cause viruses to naturally adhere to the nano-crystals.

One embodiment of the method comprises the steps of: (a) providing a dry compound that, after contact with water and a reduction in water temperature, produces nano-crystals; (b) mixing the compound with water to produce a slurry; and (c) applying the slurry to an environment that may be or is contaminated with an undesirable biological agent. After the slurry and the undesirable biological agent come into contact with one another and there is a reduction of the temperature of the water in the slurry, nano-crystals begin to form that penetrate the cell walls of the undesirable biological agents with which they are in contact, thereby neutralizing the undesirable biological agents.

In one embodiment of the method, a dry compound is utilized that, after coming into contact with water, produces a soap that weakens the cell wall of an undesirable biological agent, thereby facilitating the penetration of the cell wall by a nano-crystal.

In a further embodiment of the method, a dry compound is employed that, subsequent to being mixed with water to produce a slurry and the lowering of the temperature of the water in the slurry, produces crystals with tetrahedral shapes that have sharp edges and points that facilitate the penetration of the cell walls of undesirable biological agents. In one embodiment, the crystals are silicate crystals.

In yet another embodiment of the method, a dry compound is utilized that, subsequent to being mixed with water produces a soap that is capable of weakening the cell walls, and subsequent to the lowering of the temperature of the water in the slurry, produces nano-crystals with tetrahedral shapes. In one embodiment, the nano-crystals are silicate crystals.

In another embodiment, the water that is used in the mixing step is at a temperature that assures that upon application of the slurry to the environment, the water in the slurry will immediately begin cooling and thereby promote the rapid formation of the nano-crystals. Typically, the temperature of the water is above the ambient temperature of the environment that is to be treated.

### DETAILED DESCRIPTION

The present invention is directed to a method of neutralizing an undesirable biological agent that is or may be present in an environment which utilizes a nano-crystal to penetrate the cell wall of the undesirable biological agent and thereby neutralize the undesirable biological agent.

In one embodiment, the method comprises the steps of: (a) providing a dry compound that, after contact with water and a reduction in temperature of the water, produces nano-crystals; (b) mixing the dry compound with water to produce a slurry; and (c) applying the slurry to an environment that may be or is contaminated with an undesirable biological agent. After the slurry and the undesirable biological agent come into contact with one another and there is a reduction in the temperature of the water in the slurry, nano-crystals begin to form that penetrate the walls of the undesirable biological agents with which they are in contact, thereby neutralizing the undesirable biological agents. The undesirable biological agent can be a naturally occurring or military/weapons grade biological agent.

Environments that can be contaminated with undesirable biological agents comprise gas environments in which the agent has been dispersed, liquid environments in which the agent has been dispersed, surface environments where the agent is resident on the surface or combinations of these environments. Typically, the environments that are treated are those environments which are important to human habitation and/or cultivation. As a consequence, the gas environment is typically air and the liquid environment is typically water. Surface environments typically are the surfaces with which humans, animals or plants come into contact, as well as the surfaces of humans, animals and plants. Included within the possible surface environments are unexposed surfaces with a porous overburden or pathway that allows the liquid slurry to penetrate to the unexposed surfaces, e.g., buried soils that may harbor anthrax.

The step of providing a dry compound comprises providing a compound in which the defined constituents of the compound are in a solid state. The dry compound further has the characteristic that, after mixed with water to produce a slurry and the temperature of the water in the slurry decreases, nano-crystals form. The nano-crystals must have edges and/or points that are capable of penetrating the cell wall of a bacteria, virus and/or spore. While several forms or shapes of nano-crystals may meet this requirement, nano-crystals that have a tetrahedron shape have been found to be capable of penetrating the cell walls of bacteria. Further, while nano-crystals with tetrahedron shapes may be realized using various compounds, silicate nano-crystals have been found to produce tetrahedron with edges and/or points that are capable of penetrating the cell wall of a bacteria, virus and/or spore.

In one embodiment, the dry compound also exhibits the characteristic that, after being mixed with water, a soap is produced that is capable of weakening the cell wall of an undesirable bacteria, virus and/or spore. The soap is a sodium surfactant salt. The weakening of the cell wall facilitates the penetrating of the wall by a nano-crystal.

A dry compound that has been found to exhibit the noted silicate crystal and soap characteristics is comprised of the following compounds: sodium metasilicate (Na₂SiO₂), sodium carbonate (Na₂CO₂) and sodium sulfate (Na₂SO₄). The range of weight percentages for each of these compounds in the dry compound is:

| | |
|---|---|
| Sodium metasilicate | 5%-90% |
| Sodium carbonate | 5%-90% |
| Sodium Sulfate | 0.01%-20% |

A more preferred range of weight percentages for each of these compounds in the dry compound is:

| | |
|---|---|
| Sodium metasilicate | 40%-75% |
| Sodium carbonate | 20%-40% |
| Sodium Sulfate | 2%-5% |

The weight percentage for each of the compounds in a preferred dry compound formulation is:

| | |
|---|---|
| Sodium metasilicate | 65% |
| Sodium carbonate | 30% |
| Sodium Sulfate | 5% |

Preferably, the sodium metasilicate is in the range of 35-50 Baume and more preferably in the range of 40-41 Baume.

Further, it should be appreciated that some or all of the sodium metasilicate can be replaced with disodium trioxosilicate pentahydrate (Na₂SiO₃5H₂O) or disodium trioxosilicate anhydrate (Na₂SiO₃) and the desired characteristics still be retained. Further, some or all of the sodium sulfate can be replaced with magnesium sulfate (MgSO₄) and the desired characteristics still retained. Moreover, in certain situations, one or more of the sodium compounds can be replaced with a potassium compound.

Other materials can be added to the dry compound to achieve desired properties for a particular application. For instance, acids can be included in the dry compound for pH control. For example, after water is added to the dry compound, the pH of the resulting slurry is typically in the range of 11-13.5. If, for example, human skin is being treated and the pH of the slurry is at the upper end of the range, then acid is added to the dry compound or if appropriate, to the slurry to reduce the pH and thereby reduce skin irritation.

The dry compound is mixed with water to produce a slurry for application to an environment that is or may be contaminated with an undesirable biological agent. The slurry contains the water that provides the basis for the formation of the nano-crystals. Crystals, and in particular nanocrystals, form when the temperature of the water in which the compound is suspended decreases. Further, the crystals become longer and/or larger, when the temperature of the water decreases over a greater range. Consequently, the temperature of the water utilized in the mixing step is chosen so that upon application to the environment that is or may be contaminated, the temperature of the water will decrease a sufficient amount to promote the formation of nano-crystals that are capable of penetrating the cell walls of bacteria, viruses and/or spores. Typically, the temperature of water is greater than the ambient temperature associated with the environment that is going to be treated. Presently, the range of water temperatures that has been found to produce acceptable results extends from just above 0° C. to 200° C. . In many applications, however, a temperature range of 25°-50° C. has been found to produce acceptable results. In most known applications, a temperature range of 40-45° C. has been found to produce acceptable results. The range of ratios of the dry compound to water is 5 mg:1 l, and more preferably 250mg/1 l, and yet more preferably 1000 mg: 1 l. It should be further appreciated that the amount of water added to the dry compound can be tailored for a particular application. For instance, in certain applications, a slurry with a gel or paste consistency may be desirable. In such situations, less water is added to the compound. In other applications, a slurry with a more fluid consistency may be desirable (e.g., in applications where the slurry is to be sprayed). In such applications, more water is added to the dry compound.

After the dry compound has been mixed with water, the resulting slurry is applied to the environment that is or may be contaminated with an undesirable biological agent. The method of application depends upon the type of environment that is or may be contaminated. For instance, the slurry can be sprayed, hosed, pumped or sprinkled onto the environment. Alternatively, in certain surface environment situations, the slurry is applied by dipping the surface into a vat that holds the slurry. For example, it may be more convenient or effective to dip clothes into such a vat than to spray the clothes. In the case of gas environments, like the air, the slurry is dispersed in the environment using low-pressure atomization.

With respect to formulations of the dry compound that are used to produce a soap, it is desirable to apply the slurry within about 24 hours after mixing with water and more preferably within about 4½ hours after mixing. Thereafter, the soap begins to decompose and, as a consequence, the ability to “soften” the walls of the undesirable biological decreases.

After application of the slurry to the surface, the nano-crystals begin to form as the temperature of the water decreases. The nano-crystals that are in contact with an undesirable biological agent, provided sufficient crystal growth is achieved, penetrate the cell wall of the biological agent and thereby neutralize the biological agent. In the case of formulations of the dry compound that are used to produce a soap, the soap “softens” the cell walls, which facilitates the penetrating of the cell wall by a nano-crystal.

In the case of the formulation of the dry compound that is used to produce the silicate nano-crystals and the soap, after application of the slurry to the environment and the evaporation of the water in the slurry, any silicate crystals that were produced remain and are capable of neutralizing any new undesirable biological agents with which they come into contact for a substantial period of time, i.e., hours to days, and possibly months. In contrast, the constituents of the slurry that are not part of the nano-crystals quickly break down in an aerobic environment into sodium bicarbonates. Both the silicate nano-crystals and the sodium bicarbonates are non-toxic to humans and unlikely to be of sufficient concentration to adversely affect most other animal or plant life in the treated environment.

The dry compound is preferably produced in a two-step process. In the first step, appropriate amounts of sodium metasilicate and sodium carbonate are dry blended using a ceramic ball mill, impact blending process or other process that forcefully blends the two different particle sizes associated with sodium metasilicate and sodium carbonate to produce a common configuration, i.e., a substantially uniform grain of mesh size, that is readily available for water absorption. In the second step, the dry blend produced by the first step is allowed to cool and placed in another container. The sodium sulfate is then added to the container and loosely, dry blended with the mixture produced by the first step. It is possible to blend all three constituents of the dry compound together at the same time. However, the heat produced by the need to forcefully blend the sodium metasilicate and the sodium carbonate may cause any water associated with the mixture to dissociate and have an undesirable reaction with the sodium sulfate.

What is claimed is:

1. A method for treating an environment that may be or is known to be contaminated with an undesirable biological agent, the method comprising the acts of:
taking action to identify the environment that may be or is known to be contaminated with the undesirable biological agent;
providing a treatment substance for the environment; and
applying said treatment substance to the environment, wherein said treatment substance is capable of providing a crystal that can penetrate the cell wall of the undesirable biological agent.

2. A method, as claimed in claim 1, wherein:
said treatment substance comprises a substance capable of providing a nano-crystal.

3. A method, as claimed in claim 1, wherein:
said treatment substance comprises a silicate.

4. A method, as claimed in claim 1, wherein:
said treatment substance comprises a soluble silicate.

5. A method, as claimed in claim 1, wherein:
said treatment substance comprises sodium metasilicate.

6. A method, as claimed in claim 1, wherein:
said treatment substance comprises a soap.

7. A method, as claimed in claim 1, wherein:
said treatment substance comprises a sodium compound.

8. A method, as claimed in claim 1, wherein:
said treatment substance comprises a sodium surfactant salt.

9. A method, as claimed in claim 1, wherein:
said treatment substance comprises a slurry.

10. A method, as claimed in claim 9, wherein:
said step of applying comprises spraying said slurry onto said environment.

11. A method, as claimed in claim 9, wherein:
said step of applying comprises atomizing said slurry.

12. A method, as claimed in claim 9, wherein:
said step of applying comprises dipping said environment in a container of said slurry.

13. A method for treating an environment that may be or is known to be contaminated with an undesirable biological agent, the method comprising the acts of:
taking action to identify the environment that may be or is known to be contaminated with the undesirable biological agent;
providing a soluble compound for use in treating the environment;
mixing said soluble compound with water to produce a slurry; and
applying said slurry to the environment, wherein said slurry is capable of providing a crystal that can penetrate the cell wall of the undesirable biological agent.

14. A method, as claimed in claim 13, wherein:
said act of applying occurring when said water in said slurry is at a temperature that is above the ambient temperature of the environment.

15. A method, as claimed in claim 13, wherein:
said act of taking occurs before said act of providing.

16. A method, as claimed in claim 13, wherein:
said act of taking occurs after said act of providing.

17. A method for treating an environment that may be or is known to be contaminated with an undesirable biological agent, the method comprising the acts of:
taking action to identify the environment that may be or is known to be contaminated with an undesirable biological agent;
providing a soluble compound for use in treating the environment, wherein said soluble compound is comprised of a silicate;
mixing said soluble compound with water to produce a slurry; and
applying said slurry to the environment, wherein said slurry is capable of providing a crystal that can penetrate the cell wall of the undesirable biological agent.

18. A method, as claimed in claim 17, wherein:
said silicate is comprised of a sodium metasilicate.

19. A method, as claimed in claim 17, wherein:
said silicate is comprised of disodium trioxosilicate pentahydrate.

20. A method, as claimed in claim 17, wherein:
said silicate is comprised of disodium trioxosilicate anhydrate.

21. A method, as claimed in claim 17, wherein:
said soluble compound is comprised of sodium sulfate.

22. A method, as claimed in claim 17, wherein:
said soluble compound is comprised of magnesium sulfate.

23. A method, as claimed in claim 17, wherein:
said soluble compound is comprised of sodium carbonate.

24. A method, as claimed in claim 17, wherein:
said soluble compound is comprised of:
(a) 5%-90% by weight sodium silicate;
(b) 5%/o-90% by weight sodium carbonate; and
(c) 0.01% to 20% by weight sodium sulfate.

25. A method, as claimed in claim 17, wherein:
said soluble compound is comprised of:
(a) 40%-75% by weight sodium silicate;
(b) 20%-40% by weight sodium carbonate; and
(c) 2% to 5% by weight sodium sulfate.

26. A method, as claimed in claim 17, further comprising:
using an acid to reduce the pH of said slurry.
